(19) United States
(12) Patent Application Publication
(10) Pub. No.: US 2004/0265805 A1
(43) Pub. Date: Dec. 30, 2004

(54) Method for cloning large DNA

(75) Inventors: Qi Wang, St. Louis, MO (US); Henry E. Valentin, Wildwood, MO (US)

Correspondence Address: FULBRIGHT & JAWORSKI L.L.P., 600 CONGRESS AVE., SUITE 2400, AUSTIN, TX, 78701, US

(73) Assignee: Monsanto Technology, L.L.C.

(21) Appl. No.: 10/429,295

(22) Filed: May 2, 2003

Related U.S. Application Data
(60) Provisional application No. 60/376,979, filed on May 2, 2002.

(51) Int. Cl. C12Q 1/68; C12P 19/34

(52) U.S. Cl. 435/6; 435/91.2

(57) ABSTRACT

A method for creating large DNA vectors using DNA fragments having different but complementary restriction sites is described. The DNA vector into which the fragments are ligated may contain a clone site which is the same as one of the sites on the fragments. The invention overcomes the limitations of the prior art by allowing the creation of large DNA vector while maintaining unique cloning sites without proliferation of restriction sites, whereas prior art techniques typically increased restriction sites in a vector.

### BACKGROUND OF THE INVENTION

This application claims the priority of U.S. provisional patent application Ser. No. 60/376,979, filed May 2, 2002, the entire disclosure of which is specifically incorporated herein by reference.

1. Field of the Invention

The present invention relates to molecular biology. More specifically, the invention relates to methods and compositions for creating large DNA constructs.

2. Description of Related Art

The tools of molecular biology have enabled researchers to introduce segments of DNA from one organism to another organism. Conventional cloning methods have enabled the introduction of new pharmaceuticals and improved crops of agricultural importance. As the need for the introduction of multiple segments of DNA and larger fragments of DNA into numerous target hosts increases, the need for novel cloning strategies increases accordingly.

Typical recombinant DNA techniques involve steps for isolating, analyzing and manipulating the DNA in vitro prior to introducing the DNA into a target host. Typically, the DNA of the donor vector or donor genome is prepared. Restriction enzymes, also referred to as restriction endonucleases, are used to cut the donor DNA at specific locations usually called cloning sites. Different restriction enzymes recognize different sequences of nucleotides on the DNA and cleave the DNA polymer at these sequences. DNA fragments are then isolated and ligated into a cloning vector. The ligation process relies on the use of another enzyme (DNA ligase) that can bond segments of DNA together.

A cloning vector is a nucleic acid molecule into which DNA fragments can be introduced in vitro using the restriction enzymes and DNA ligases. A number of cloning vectors exist, including, but not limited to, plasmids and bacteriophages. Different types of cloning vectors can be used, depending on the target host organism into which the DNA is introduced.

One of the goals of genetic engineering is to produce hosts with important characteristics or traits. Recent advances in genetic engineering have provided the requisite tools to transform hosts to contain and express foreign genes (Kahl et al., World J. Microbiol.Biotech., 11:449-460, 1995). Particularly desirable traits of interest for genetic engineering would include but are not limited to protein production, resistance to insects and other pests and disease-causing agents, tolerances to herbicides, enhanced stability, yield, or shelf-life, environmental tolerances, and nutritional enhancements.

The technological advances in transformation and regeneration have enabled researchers to take segments of DNA, such as a gene or genes from a heterologous source, or a native source, but modified to have different or improved qualities, and incorporate the exogenous DNA into a host's genome. The gene or gene(s) can then be expressed in the host cell to exhibit the added characteristic(s) or trait(s). In most transformation approaches, a single vector containing 1-2 genes conferring desirable characteristic(s) is introduced into a host of interest via an appropriate expression vector.

Conventional strategies for introducing multiple genes into hosts of interest are time-consuming and labor intensive. For example, to introduce multiple genes of interest into a target plant requires the introduction of the plant expression vector and subsequent screening of the transformed plants for the desired characteristic(s), followed by a second transformation event into a parent plant generated by the first step, with subsequent screening of the second generation of transformed lines for the desired characteristic(s) conferred by the gene(s) on the second expression construct. This method can be a time intensive process, essentially doubling or tripling the time and labor it takes to generate a plant with desirable characteristics in a conventional cloning method using multiple purification steps and restriction enzymes.

Manipulation with large fragments of DNA using conventional cloning techniques is even more challenging and thus there is a great need in the art for more efficient methods of introducing large, multigene cassettes into target plants of interest. Current methods of constructing large (over 20 kilobases) cloning vectors (herein referred to as megavectors), are extremely inefficient.

One of the limitations of conventional cloning strategies is that large DNA fragments are often difficult to clone because the fragments contain multiple internal restriction sites that limit the number of usable restriction enzymes for the cloning process. Thus, special vectors must be designed and rare restriction enzymes may be necessary to clone large segments of DNA because of the lack of a unique cloning site.

Due to the lack of unique cloning sites it is very difficult to produce vectors larger than 20 kbp from smaller DNA fragments in a predictable manner. Vectors larger than 30 kbp are close to impossible to generate without having a proper strategy in place. Since it is expected that future biotechnological projects will become more complicated than in the past and potentially will require multiple genes to be expressed as transgenes, it will be critical to have a strategy as described above in place.

Consequently, a novel cloning strategy that provides for a unique cloning site after each fragment is ligated and that can be used to clone large size DNA fragments would be more efficient, less labor intensive, and an improvement over existing cloning methods. What is needed is a strategy to construct mega vectors (>20 kbp) using multiple DNA fragments or even cassettes, in which restriction sites are not proliferated and unique cloning sites can be maintained.

### SUMMARY OF THE INVENTION

In one aspect, the invention provides a method for cloning large nucleic acid constructs (mega vectors) comprising the steps of: a) providing a first polynucleotide (a DNA cassette or fragment) having different first and second restriction sites, said sites having complementary first and second overhangs when cleaved; b) providing a second polynucleotide (a vector) having a unique cloning site comprising a restriction site which is the same as that of the first restriction site of the first polynucleotide; c) cleaving said second polynucleotide at the cloning site to create first and second overhangs on the ends of the second polynucleotide; d) ligating said first polynucleotide into said second polynucleotide at the cleaved cloning site, thereby recreating said cloning site through the ligation product of the first overhang of the first polynucleotide and first overhang of the second polynucleotide; and e) repeating steps a) through d) until the construct is complete.

In another aspect, the invention provides a method for preparing a nucleic acid construct comprising the steps of: a) providing a first polynucleotide having a first overhang for a first restriction site and a second overhang for a different second restriction site, wherein said first and second overhangs are complementary; b) providing a second polynucleotide having overhangs for a unique cloning site comprising a restriction site which is the same as that of the first restriction site of the first polynucleotide; and c) ligating said first polynucleotide into said second polynucleotide at the overhangs for the unique cloning site, thereby recreating a cloning site through the ligation product of the first overhang of the first polynucleotide and first overhang of the second polynucleotide with the overhangs of the unique cloning site. The method may further comprise repeating steps a) through c) until the construct is complete. The construct may be at least 20 or 30 kb in size. The first polynucleotide and/or the second polynucleotide may comprise any desired elements, including a coding sequence and a regulatory element. The overhangs may be prepared by contacting a starting polynucleotide with a restriction enzyme.

In certain embodiments, of the invention, a pair of restriction sites are used selected from the group consisting of Not I and Bsp 120I, AscI and MluI, and SbfI and Nsi I, or isoschizomers thereof. In further embodiments, the first restriction site is a Not I restriction site, and the second restriction site is a Bsp 1201, BseX3I, BstZ I, EagI, PspOMI, or Xma III restriction site. In still further embodiments, the first restriction site is an AscI restriction site, and the second restriction site is a MluI, MluI, BspPI, BssHII, Paul, BsaJI, Asc I or Mlu I restriction site. In yet another embodiment, the first restriction site is an SbfI restriction site, and the second restriction site is selected from the group consisting of a Mph1103I, NsiI, PstI, or Zsp2I restriction site. In one aspect, the first and/or second restriction site is 6 bp and/or 8 bp.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a textual illustration of NotI/Bsp120I compatibility and cloning of the inventive method.

FIG. 2 is a vector map illustrating NotI/Bsp 120I cloning of the inventive method.

FIG. 3 is a textual illustration of sticky end compatibility of Asc I with Mlu I and BssH II.

FIG. 4 is a textual illustration of sticky end compatibility of Sbf I with Nsi I and Pst I.

FIG. 5 illustrates various possible constructs of multi-gene vectors.

FIG. 6 is a textual illustration of the primers used to prepare vector pMON36582.

FIG. 7 is the shuttle vector designated pMON36582.

FIG. 8 shows a shuttle vector pMON36586.

FIG. 9 illustrates the use of shuttle vectors and binary vectors to construct expression cassettes and mega vectors.

FIG. 10 is a Pullex map of pMON36596.

FIG. 11 is a Pullex map of pMON36597.

FIG. 12 is a Pullex map of pMON77602.

FIG. 13 is a Pullex map of pMON77601.

FIG. 14 is a Pullex map of pMON36582.

FIG. 15 is a Pullex map of pMON69943.

FIG. 16 is a Pullex map of pMON69929.

FIG. 17 is a Pullex map of pMON69936.

FIG. 18 is a Pullex map of pMON36592.

FIG. 19 is a Pullex map of pMON69945.

### DESCRIPTION OF ILLUSTRATIVE EMBODIMENTS

The invention provides procedures for assembling mega vectors. When limited to standard cloning procedures, it becomes increasingly difficult to add DNA-fragments, including cassettes, to increasingly large vectors, especially those that exceed 20 kbp in size. In most cases this is due, at least in part, to the lack of unique cloning sites within such a construct due to the propagation of restriction sites. Theoretically, this can be overcome to some extent by using large poly cloning sites which contain a large number of unique restriction sites. However, as the vector grows, the number of unique cloning sites available continues to diminish.

Most poly linkers utilize restriction enzymes that recognize 6 bp sites. These sites occur randomly in native DNA, on average, every 4096 bp. Thus, for every polynucleotide inserted into a vector, using known methods, additional cloning sites are necessarily added at predictable intervals based on probability. Moreover the restriction sites of the DNA fragment to be added, recreate additional restriction sites in the vector once the fragment is ligated into the vector polynucleotide.

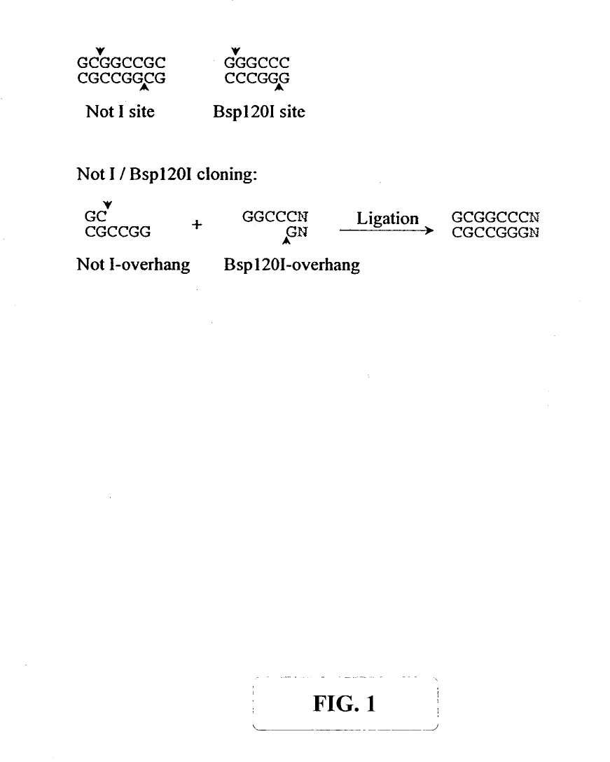

The present invention, in one embodiment, comprises use of different yet ligation-compatible restriction sites on the ends of DNA fragments, providing the significant benefit of inhibiting the addition of such restriction sites. As shown in FIG. 1, the use of the different, yet ligation compatible, restriction sites (Not I and Bsp 1201 in this example) on the ends of the DNA fragment, along with the use of one of the same sites for the cloning site on the vector, means that the ligated junction of the dissimilar but compatible sticky-ends is not cleavable by either restriction enzyme.

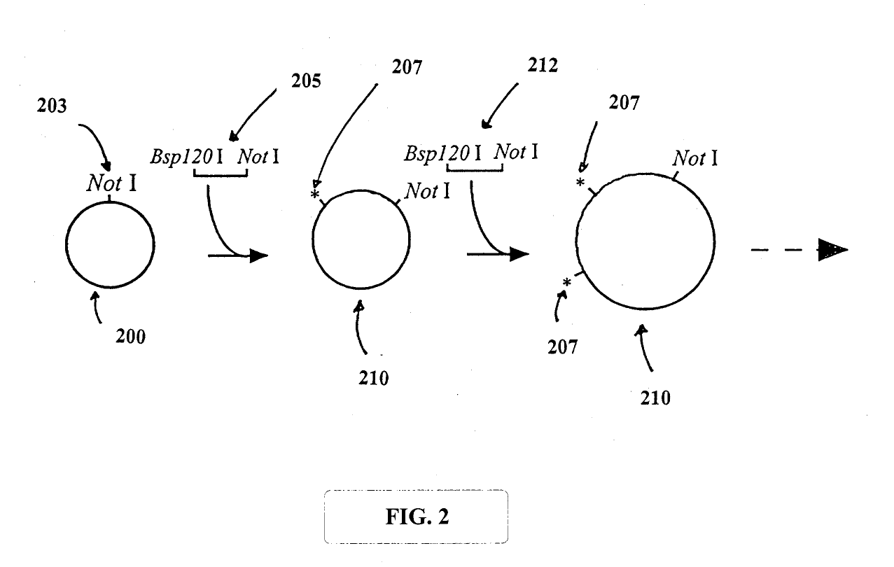

In one experimental setup using a vector with a unique 8 bp cloning site and DNA-cassettes flanked by a Not 18 bp recognition site plus a compatible site such as Bsp120I, a cloning method can be developed which allows the assembly of very large vectors. FIG. 2 illustrates this example of a cloning method. The DNA fragment 205 has different yet complementary ends once prepared for cloning. The vector 200 contains a cloning site 203 comprising a restriction site which is the same as one of the two sites terminating the fragment 205. In FIG. 2, the common site is Not I. Once the vector 200 is cleaved at the cloning site, the fragment 205 is ligated into the vector to create the mega vector 210. Additional fragments 212 may then be inserted as necessary to complete the mega vector construct 210. Sites 207, labeled by an asterisk, indicate Not I/Bsp 120I sites that cannot be cleaved by either Not I or Bsp120I. As a result, the growing mega vector 210 continues to harbor a unique Not I site and the procedure can be repeated indefinitely. In so doing, the cloning site 203 remains unique.

Additional embodiments of the invention are exemplified employing additional complementary pairs of restriction enzymes. Other examples of sites compatible with the NotI overhang are BseX3I, BstZ I, EagI, PspOMI, Xma III. An overhang is a sequence corresponding to the cleaved product of a given restriction site. An advantage of using Bsp120I is that upon ligation of the compatible ends, none of the enzymes used to generate these ends can recleave the resulting sequence. Examples of other pairings with AscI may be used which produce compatible ends with MluI, BspPI, BssHII, Paul, BsaJI, and other enzymes; CciNI (an isochizomer of NotI); SbfI (and its isochizomers SdaI, Sse8387I) which are compatible with Mph1103I, NsiI, PstI, Zsp2I, and other enzymes.

The 8 bp cutters AscI and Not I may have preferred restriction sites in certain embodiments, because the overhangs produced by these enzymes consist of guanine and cytosine only. These bases produce 8 hydrogen bonds with their counterparts, and therefore provide an increased ligation efficiency compared to enzymes which produce overhangs with adenine and thymidine overhangs.

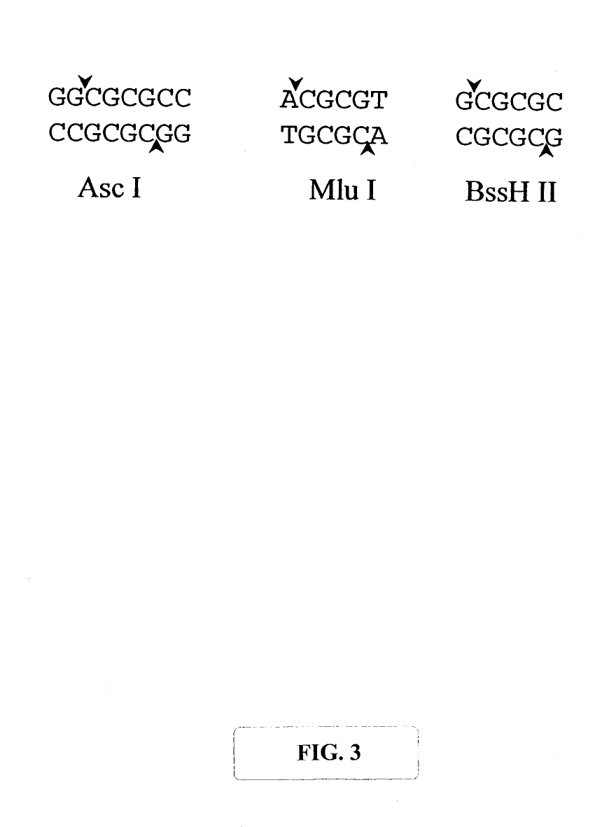

FIG. 3 illustrates an example of the use of Asc I as an eight base pair cutter. Mlu I and BssH II restriction enzymes produce compatible ends to Asc I. Only the ligation product Mlu I overhangs with Asc I overhangs results in a sequence that can not be recleaved by either enzyme. For this reason, a cloning system using Asc I and Mlu I is a preferred system in certain embodiments, although a system using Asc I and BssH II or other enzymes producing compatible overhangs may be used as well.

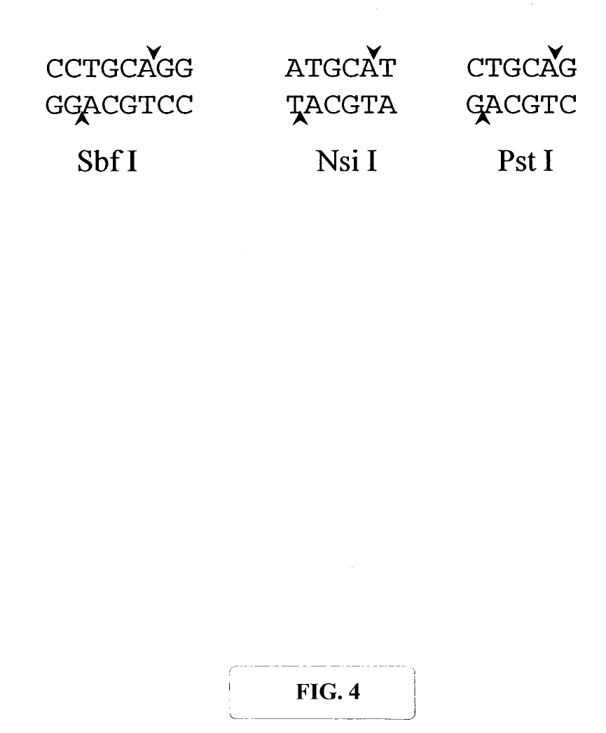

FIG. 4 illustrates an example of the use of the Sbf I enzyme. Utilization of the restriction enzyme pair Sbf I and Nsi I results in ligation products which cannot be recleaved by either enzyme.

Another example of an embodiment of the invention utilizes two restriction enzymes with a recognition sequence of 8 bp, which produce compatible ends and cannot be recleaved by either enzyme. Yet another use of an enzyme pair with an even larger recognition site producing compatible ends, which cannot be recleaved upon ligation. The larger the recognition site, the lower the probability that such a site occurs naturally within the sequence to be cloned. While a 6 bp recognition site occurs on average every 4⁶ bp (=4096 bp), an 8 bp recognition site occurs naturally only every 4⁸ bp (=65536 bp). The less likely a natural occurrence of the utilized restriction enzyme recognition sites is, the greater the ease of cloning additional DNA-fragments into a growing vector using these enzymes.

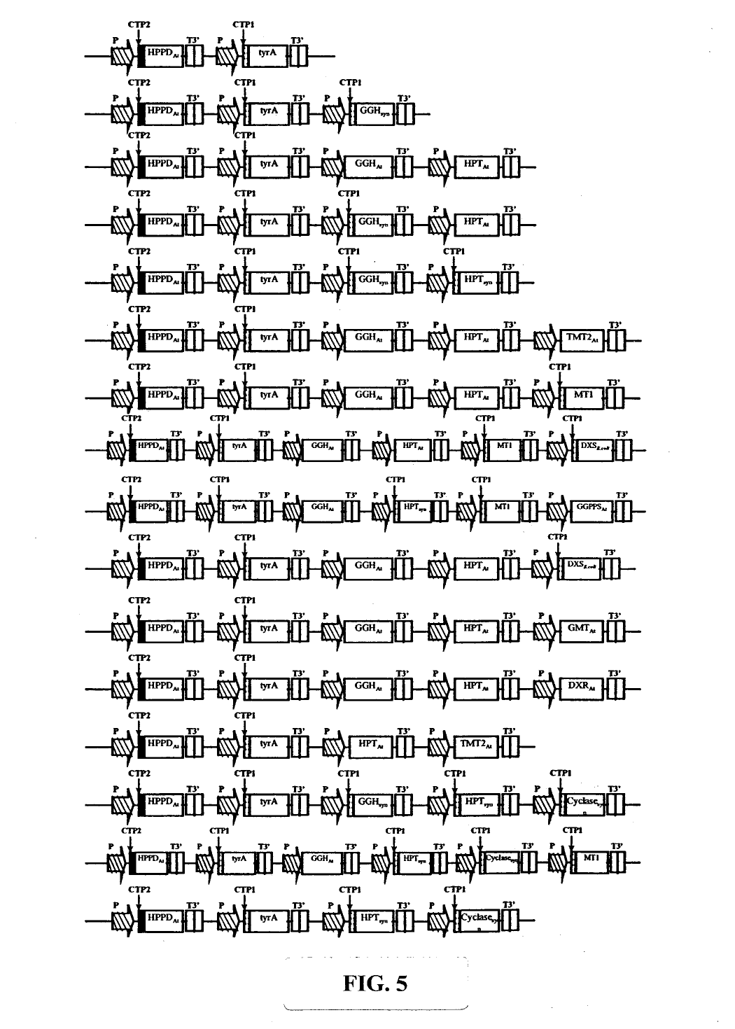

The first polynucleotide used in the invention may be a DNA fragment, gene, gene fragment or any other DNA structure, including a gene expression cassette comprising a promoter, for example, the Napin promoter, a terminator, a plastid target peptide (which can be the natural plastid target peptide, or a N-terminal fused chloroplast target peptide), and a gene of interest. FIG. 5 illustrates various such cassettes. The expression cassettes can be oriented head to tail as in FIG. 5, head to head, or the orientation can vary.

A vector prepared in accordance with the invention may be transformed into a host cell by any desired method. In certain embodiments of the invention, the host cell may be a prokaryotic or eukaryotic cell and may further be, for example, a plant, animal, bacterial, yeast or fungal cell. Suitable methods for transformation of such cells that may be used with the invention are well known to those of skill in the art and are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, and 5,780,448), including microinjection (U.S. Pat. No. 5,789,215); by electroporation (U.S. Pat. No. 5,384,253); by calcium phosphate precipitation; by using DEAE-dextran followed by polyethylene glycol; by direct sonic loading; by liposome mediated transfection and receptor-mediated transfection; by microprojectile bombardment (PCT Application Nos. WO 94/09699 and 95/06128; U.S. Pat. Nos. 5,610,042; 5,322,783 5,563,055, 5,550,318, 5,538,877 and 5,538,880); by agitation with silicon carbide fibers (U.S. Pat. Nos. 5,302,523 and 5,464,765); by PEG-mediated transformation of protoplasts (U.S. Pat. Nos. 4,684,611 and 4,952,500; by desiccation/inhibition-mediated DNA uptake, and any combination of such methods. Through the application of techniques such as these, organelle(s), cell(s), tissue(s) or organism(s) may be stably or transiently transformed.

### EXAMPLES

### EXAMPLE 1

### Construction of a Multi-Gene Mega Vector and DNA Cassette

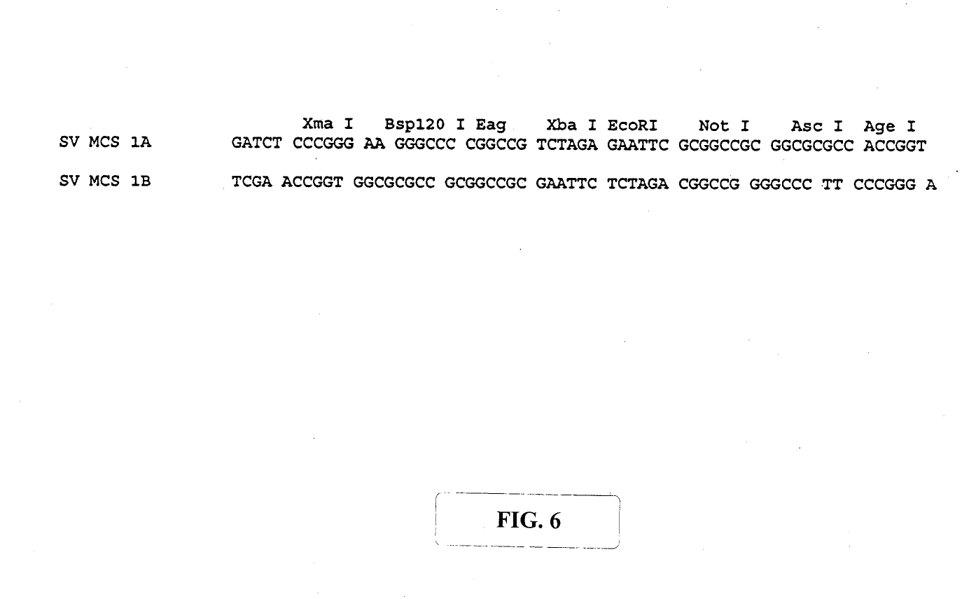

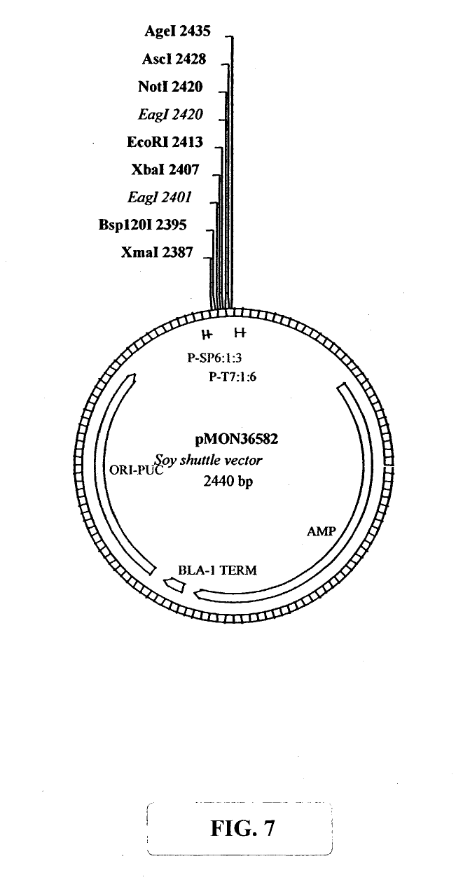

Cloning is performed using expression cassettes flanked by Bsp120 I (GGGCCC) and Not I (GCGGCCGC) restriction sites as shown in FIG. 6. A shuttle vector was constructed by annealing primers SV MCS 1A and SV MCS 1B and ligating them into Bgl II and Xho I digested and gel purified pSP72 (Promega, www.promega.com). The resulting mega vector was designated pMON36582, FIG. 7, and was confirmed by DNA sequencing.

All gene expression cassettes used were set up to be flanked by Not I restriction sites. These cassettes were isolated by digesting the previous vectors with Not I, followed by gel purification of the expression cassettes. pMON36582 was digested with Eag I, which cuts twice in this vector, once within the Not I site, and once 19 bp upstream of the Not I site. Both overhangs were compatible with Not I. The Not I expression cassettes were ligated into gel purified Eag I digested pMON36582, resulting in a vector with a single Not I site only. As a result of this cloning, the expression cassette is now available as a Bsp120 I/Not I cassette.

### EXAMPLE 2

### Expression Cassette for the Arabidopsis homogentisate phytyltransferase available as a Bsp120 I/Not I cassette

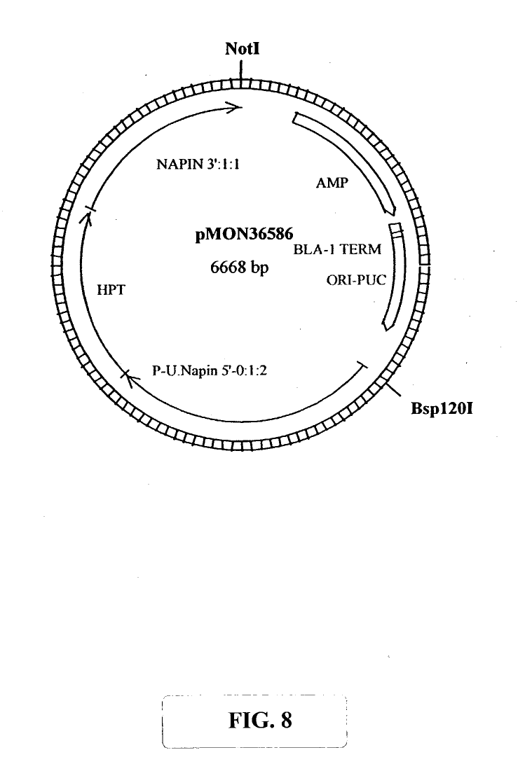

FIG. 8 shows an example of a shuttle vector harboring an expression cassette of the Arabidopsis homogentisate phytyltransferase (HPT) as a Bsp120 I/Not I cassette. The Napin promoter and napin terminator were fused to the 5′ and 3′ ends, respectively to drive seed specific expression. This vector and cassette were obtained as described in Example 1.

### EXAMPLE 3

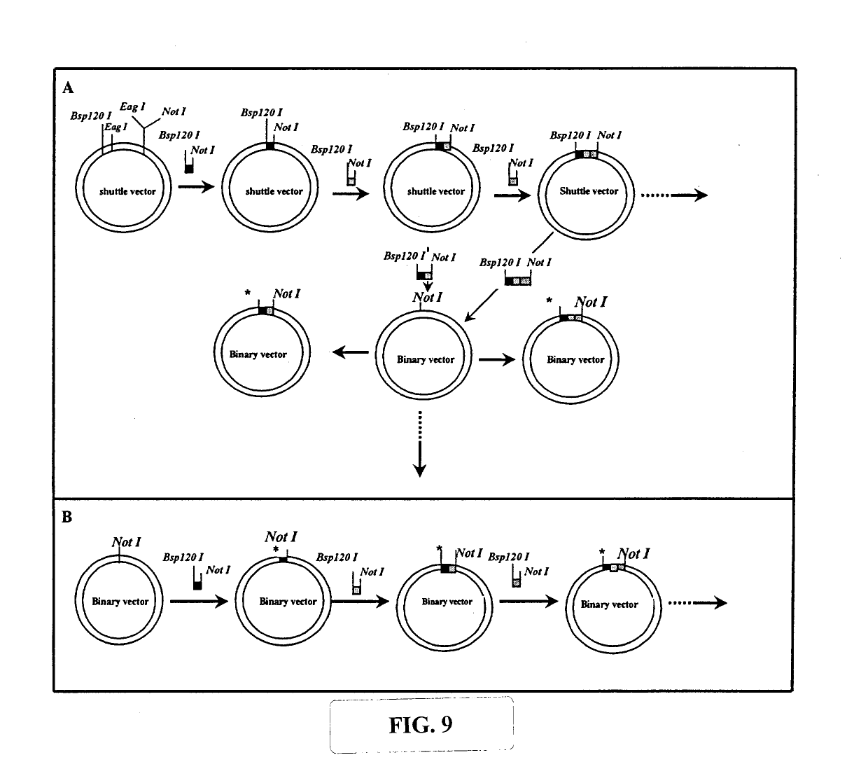

Assembly of gene expression cassettes in a shuttle or binary vector FIG. 9 shows each gene expression cassette containing a promoter, a 5′ untranslated region, a gene of interest, and a 3′ untranslated region. If desired, other elements such as introns, chloroplast target peptides (CTPs) can be included into the gene expression cassettes as well.

The assembly of expression cassettes can now be performed in a shuttle vector, such as pMON36586 (see also FIG. 9A). Gene expression cassettes are released from other shuttle vectors by Bsp120 I/Not I digests, and ligated into a shuttle vector such as pMON36586 which has been digested with Not I. The resulting vector harbors one additional gene expression cassette and a single Not I site. This procedure can be repeated as required. Upon completion of the gene assembly, the combined expression cassettes can be released by Bsp120 I/Not I digest (FIG. 9A). The resulting fragment carrying the expression cassettes can then be purified and ligated into a single Not I site of a binary vector (FIG. 9A).

Alternatively, the assembly of gene expression cassettes can be performed directly in a binary vector (FIG. 9B). A binary vector is defined by the presence of the right and left border sequences, which are necessary for DNA transfer from Agrobacterium into plant cells.

All chemical reagents and enzymes for these examples are molecular grades. These reagents and enzymes were utilized according to the supplier's instructions. Standard molecular cloning techniques were used.

### EXAMPLE 4

### Construction of Binary Vectors for Tocopherol Biosynthesis

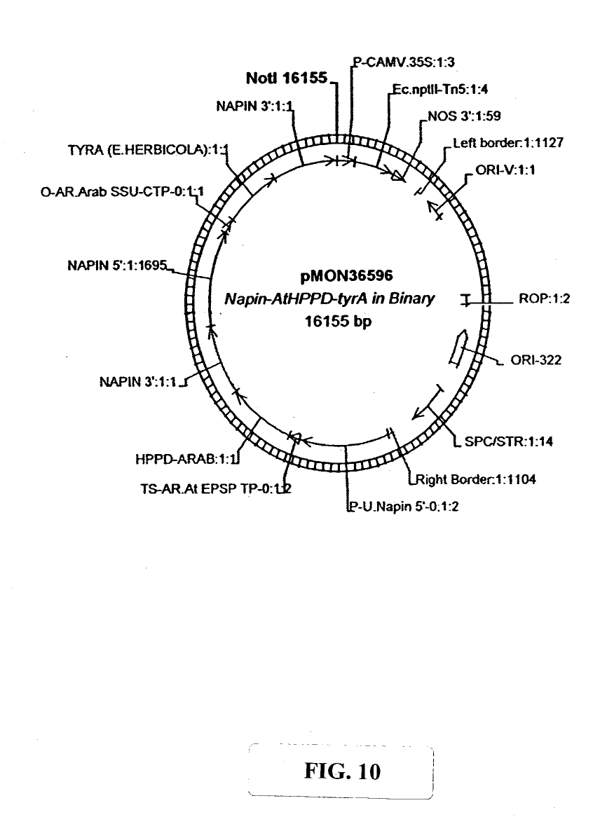

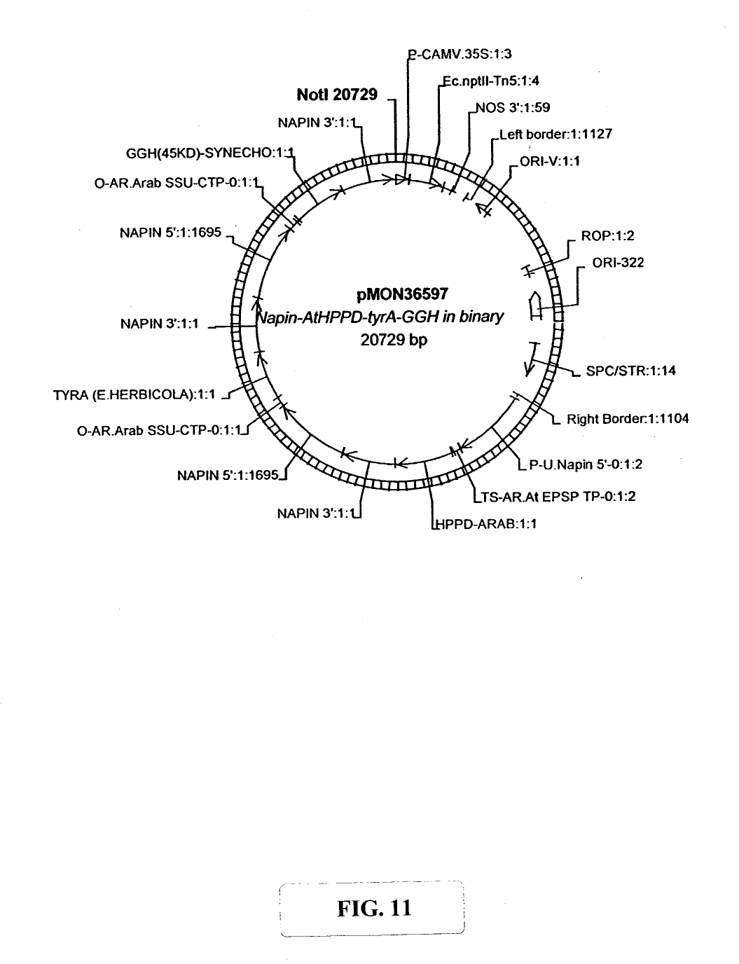

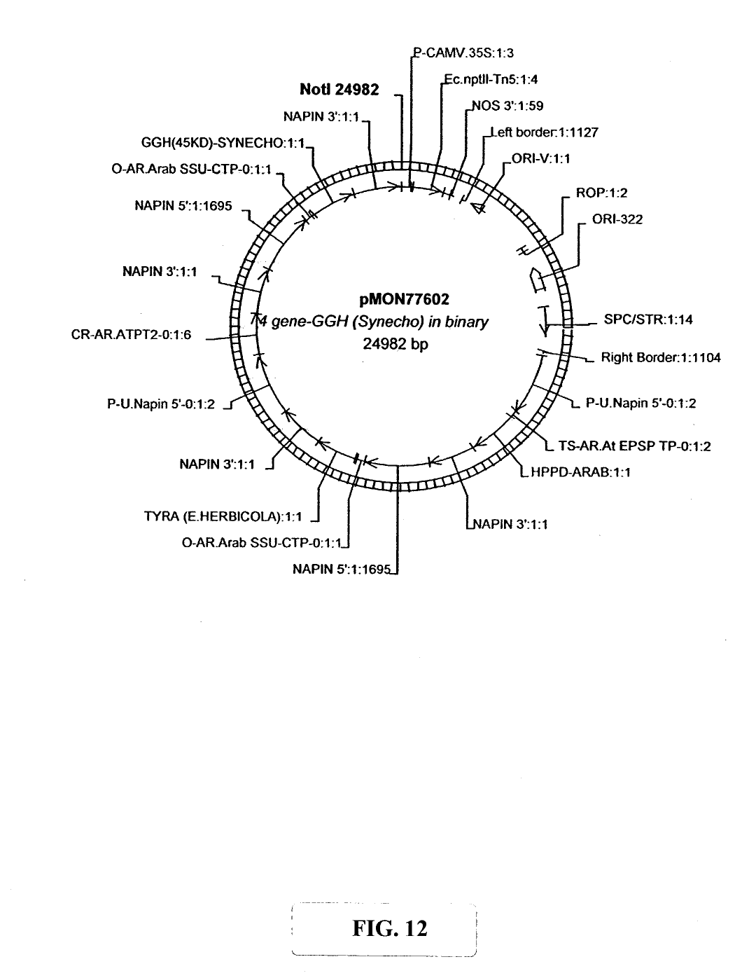

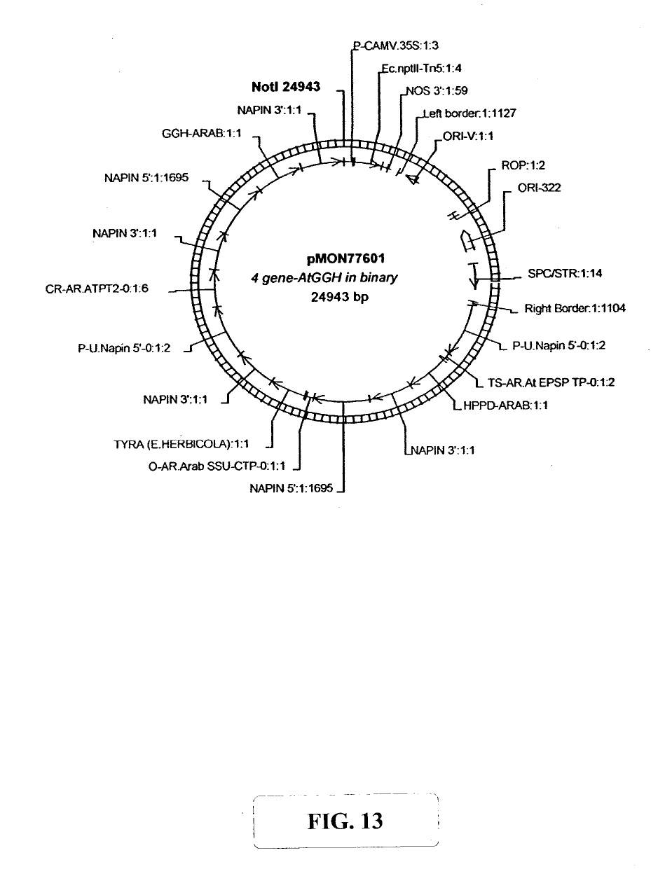

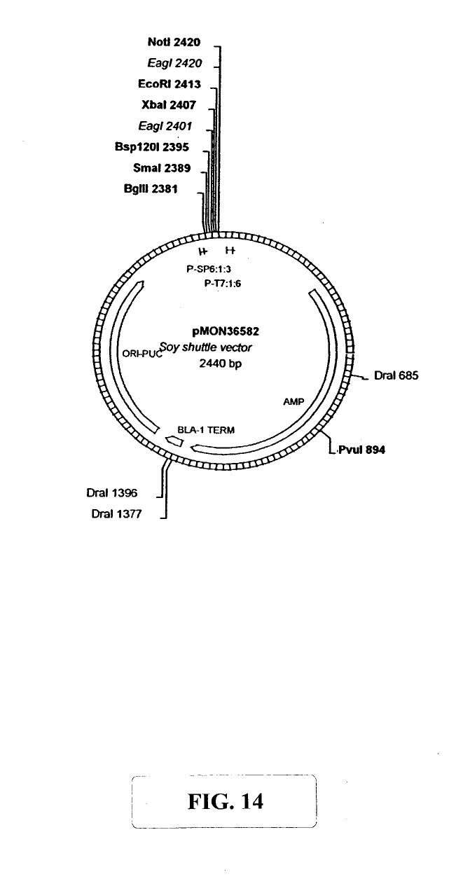

The binary vectors containing tyrA combinations with other genes of interest for tocopherol pathway engineering are listed in FIG. 5. Components of these constructs are also provided in the Table I. The Pullux maps at FIGS. 10, 11, 12, 13 and 14 supply more detail information about these constructs.

TABLE 1
List of binary vectors to be transformed into Arabidopsis thaliana
to engineer tocopherol biosynthesis

| PmoN # | Gene Combination | Genetic Elements |
|---|---|---|
| 36596 | HPPD_{At}/tyrA | Napin 5′ & Napin 3′; |
| | | CTP1&2 |
| 36597 | HPPD_{At}/tyrA/GGH_{syn} | Napin 5′ & Napin 3′; |
| | | CTP1&2; native CTPs |
| 77601 | HPPD_{At}/tyrA/GGH_{At}/HPT_{At} | Napin 5′ & Napin 3′; |
| | | CTP1&2; native CTPs |
| 77602 | HPPD_{At}/tyrA/GGH_{syn}/HPT_{At} | Napin 5′ & Napin 3′; |
| | | CTP1&2; native CTPs |
| 66657 | HPPD_{At}/tyrA/GGH_{syn}/HPT_{syn} | Napin 5′ & Napin 3′; |
| | | CTP1&2; native CTPs |
| 66659 | HPPD_{At}/tyrA/GGH_{At}/HPT_{At}/TMT2 | Napin 5′ & Napin 3′; |
| | | CTP1&2; native CTPs |
| | HPPD_{At}/tyrA/GGH_{syn}/HPT_{At}/MT1 | Napin 5′ & Napin 3′; |
| | | CTP1&2; native CTPs |
| | HPPD_{At}/tyrA/HPT_{At}/TMT2 | Napin 5′ & Napin 3′; |
| | | CTP1&2; native CTPs |
| | HPPD_{At}/tyrA/GGH_{At}/HPT_{At}/MT1/DxS | Napin 5′ & Napin 3′; |
| | | CTP1&2; native CTPs |
| | HPPD_{At}/tyrA/GGH_{At}/HPT_{At}/DxS_{E. coli} | Napin 5′ & Napin 3′; |
| | | CTP1&2; native CTPs |
| | HPPD_{At}/tyrA/GGH_{At}/HPTsyn/MT1/ | Napin 5′ & Napin 3′; |
| | GGPPS At | CTP1&2; native CTPs |
| | HPPD_{At}/tyrA/HPT_{At}/GGH_{At}/DxR_{At} | Napin 5′ & Napin 3′; |
| | | CTP1&2; native CTPs |
| | HPPD_{At}/tyrA/GGH_{At}/ | Napin 5′ & Napin 3′; |
| | HPT_{syn}/Cyclase_{syn}/MT1 | CTP1&2; native CTPs |
| | HPPD_{At}/tyrA/GGH_{syn}/HPT_{syn}/ | Napin 5′ & Napin 3′; |
| | Cyclase_{syn} | CTP1&2; native CTPs |
| | HPPD_{At}/tyrA/HPT_{Syn}/Cyclase_{syn} | Napin 5′ & Napin 3′; |
| | | CTP1&2; native CTPs |
| | HPPD_{At}/tyrA/GGH_{At}/HPT_{At}/GMT_{At} | Napin 5′ & Napin 3′; |
| | | CTP1&2; native CTPs |

### EXAMPLE 5

### Glycine Max Transformation with tyrA Gene Combinations

This example describes using the large vector production method in preparation of plant binary vectors to test tyrA in combination with other key enzymes in the tocopherol biosynthetic pathway to enhance tocopherol production in transgenic Glycine max seeds.

The table II describes the plant binary vectors prepared for G. max transformation with their respective gene of interest expression cassettes for seed-specific expression of the transgenes.

TABLE 2
List of constructs transformed to G. max.

| Construct | |
|---|---|
| number | Genetic elements |
| PMON69943 | p7Sα′::CTP2::HPPD Arabidopsis::E9 3′/p7Sα′::CTP1:: |
| | tyrA_{E. herbicola}::E9 3′/parcelin-5::CTP1:: |
| | HPT_{Synchocystis}::Arcelin 3′ |
| PMON69945 | p7Sα′::CTP2::HPPD Arabidopsis::E9 3′/p7Sα′::CTP1:: |
| | tyrA_{E. herbicola}::E9 3′/parcelin-5::CTP1::HPT_{Synchocystis}:: |
| | Arcelin 3′/pNapin::GGH_{Arabidopsis}::napin 3′ |

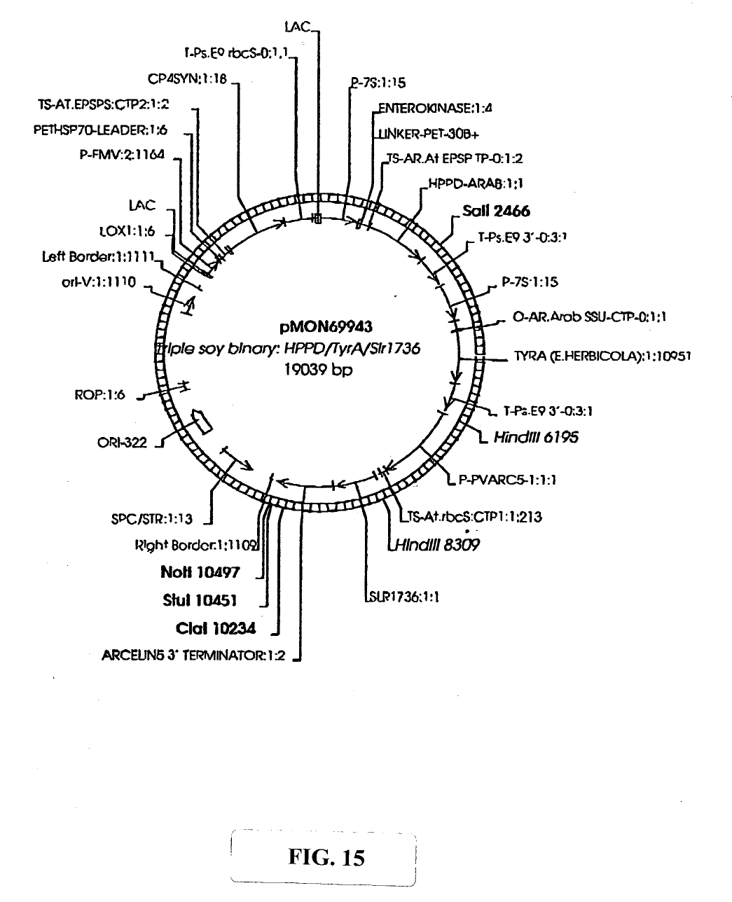

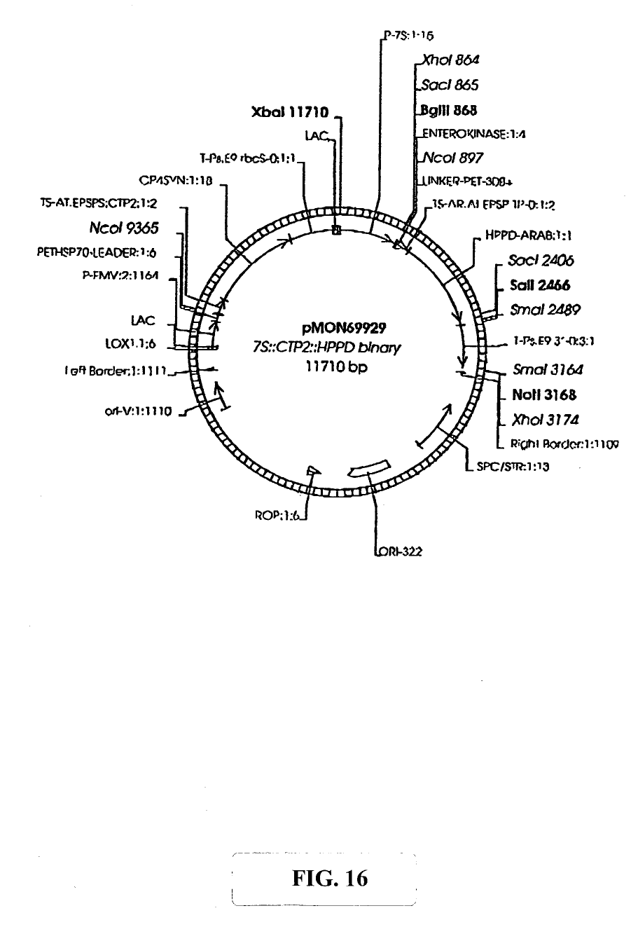

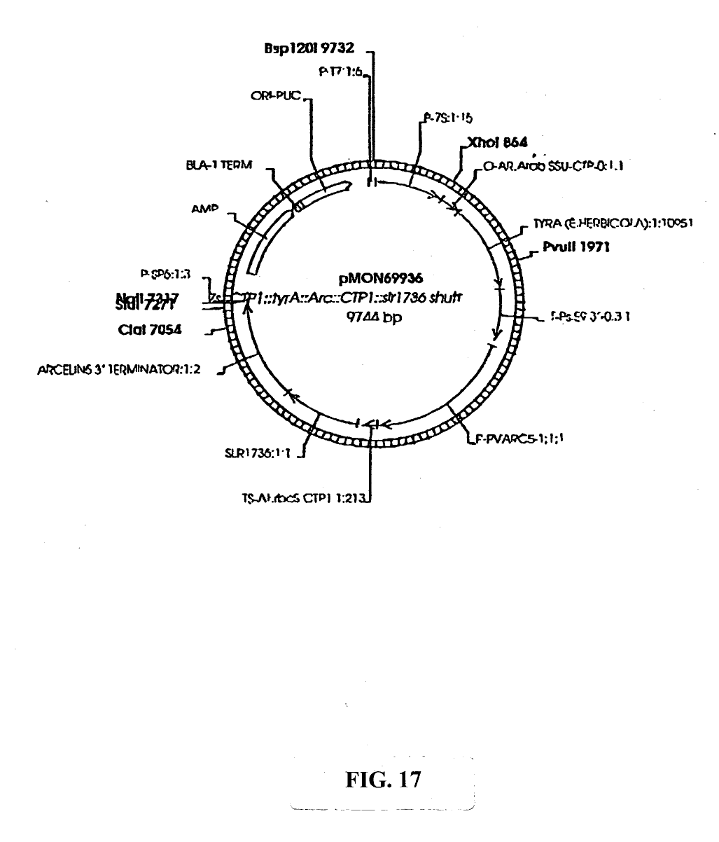

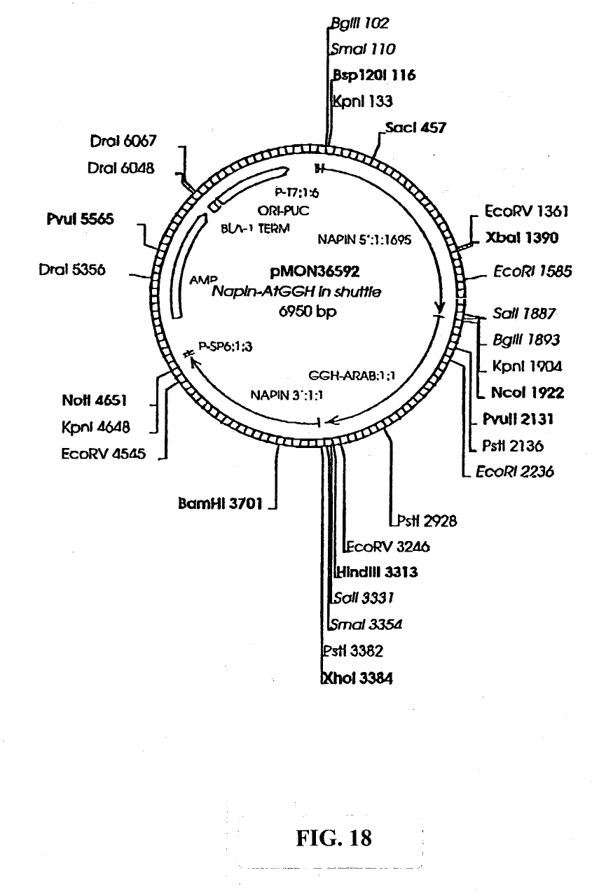

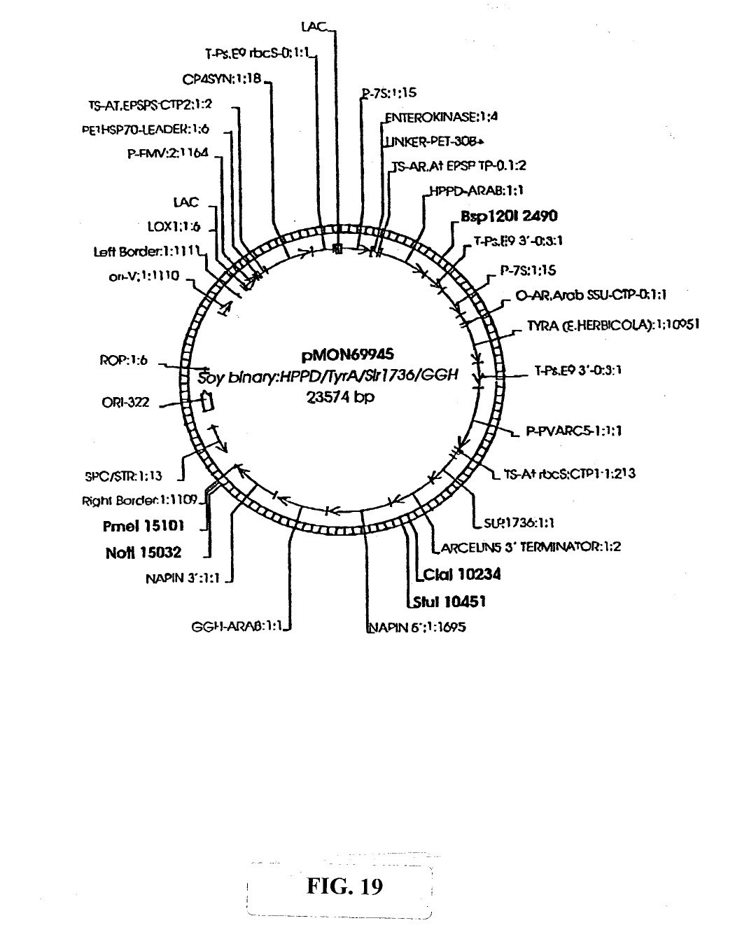

The plant binary vector pMON69943 (FIG. 15) was prepared by digesting the pMON69929 (FIG. 16), containing the p7Sα⁷::CTP2::HPPD_{Arobidopsis}::E9 3′ expression cassette, with Not I and ligating with 7.3 kb gel purified fragment generated by digestion of pMON69936 (FIG. 17) with Bsp120I and NotI. This fragment contains the expression cassettes of p7Sα′::CTP1::tyrAE. _{herbicola}::E9 3′ and pArcelin-5::CTP1::HPT_{synechocystis}::Arcelin 3′. The pMON69943 was further digested with NotI and ligated with 4.5 kb gel purified Bsp120I/NotI fragment from pMON36592 (FIG. 18) to generate the pMON69945 (FIG. 19). The fragment from pMON36592 contains the expression cassette of pNapin::GGH_{Arabidopsis}::napin 3′.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

What is claimed is:

1. A method for preparing a nucleic acid construct comprising the steps of:
a) providing a first polynucleotide having a first overhang for a first restriction site and a second overhang for a different second restriction site, wherein said first and second overhangs are complementary;
b) providing a second polynucleotide having overhangs for a unique cloning site comprising a restriction site which is the same as that of the first restriction site of the first polynucleotide; and
c) ligating said first polynucleotide into said second polynucleotide at the overhangs for the unique cloning site, thereby recreating a cloning site through the ligation product of the first overhang of the first polynucleotide and first overhang of the second polynucleotide with the overhangs of the unique cloning site.

2. The method of claim 1, wherein steps a) through c) are repeated at least once until the construct is complete.

3. The method of claim 1, wherein the nucleic acid construct is at least 20 kb in size.

4. The method of claim 1, wherein the nucleic acid construct is at least 30 kb in size.

5. The method of claim 1, wherein the first polynucleotide and/or the second polynucleotide comprise a coding sequence.

6. The method of claim 1, wherein the first polynucleotide and/or the second polynucleotide comprise a regulatory element.

7. The method of claim 1, wherein the step of providing a first polynucleotide comprises contacting a starting polynucleotide with restriction enzymes that specifically cleave the first and second restriction sites.

8. The method of claim 1, wherein the step of providing a second polynucleotide comprises contacting a starting polynucleotide with a restriction enzyme that specifically cleaves the unique cloning site.

9. The method of claim 1, wherein the first and second restriction sites are a pair of restriction sites selected from the group consisting of Not I and Bsp 120I, AscI and MluI, EaeI and SbfI and Nsi I, or isoschizomers thereof.

10. The method of claim 1, wherein the first restriction site is a Not I restriction site, and the second restriction site is a Bsp 120I, BseX3I, BstZ I, EagI, PspOMI, or Xma III restriction site.

11. The method of claim 1, wherein the first restriction site is an AscI restriction site, and the second restriction site is a MluI, BspPI, BssHII, Paul, or BsaJI restriction site.

12. The method of claim 11, wherein the first restriction site is an AscI restriciton site and and the second restriction site is a MluI restriction site.

13. The method of claim 1, wherein the first restriction site is an SbfI restriction site and the second restriction site is an Nsi I restriction site.

14. The method of claim 1, wherein the first restriction site is an SbfI restriction site, and the second restriction site is a Mph1103I, NsiI, PstI, or Zsp2I restriction site.

15. The method of claim 1, wherein the first and/or second restriction site is 6 bp.

16. The method of claim 1, wherein the first and/or second restriction site is 8 bp.

17. A method for preparing a nucleic acid construct comprising the steps of:
a) providing a first polynucleotide having different first and second restriction sites, said sites having complementary first and second overhangs when cleaved;
b) providing a second polynucleotide having a unique cloning site comprising a restriction site which is the same as that of the first restriction site of the first polynucleotide;
c) cleaving the first polynucleotide at the first and second restriction sites to create first and second overhangs of the first polynucleotide and cleaving said second polynucleotide at the cloning site to create first and second overhangs on the ends of the second polynucleotide; and
d) ligating said first polynucleotide into said second polynucleotide at the cleaved cloning site, thereby recreating said cloning site through the ligation product of the first overhang of the first polynucleotide and first overhang of the second polynucleotide.

18. The method of claim 17, wherein steps a) through d) are repeated at least once until the construct is complete.

19. The method of claim 17, wherein the nucleic acid construct is at least 20 kb in size.

20. The method of claim 17, wherein the nucleic acid construct is at least 30 kb in size.

21. The method of claim 17, wherein the first polynucleotide and/or the second polynucleotide comprise a coding sequence.

22. The method of claim 17, wherein the first polynucleotide and/or the second polynucleotide comprise a regulatory element.

23. The method of claim 17, wherein the first and second restriction sites are a pair of restriction sites selected from the group consisting of Not I and Bsp 120I, AscI and MluI, and SbfI and Nsi I, or isoschizomers thereof.

24. A method for preparing a nucleic acid construct comprising the steps of:
a) providing a first polynucleotide comprising a first restriction site for a first restriction enzyme and a second polynucleotide comprising a second restriction site for a second restriction enzyme, wherein the first restriction site is compatible with the second restriction site;
b) contacting the first polynucleotide with the first restriction enzyme and the second polynucleotide with the second restriction enzyme; and
c) ligating the first polynucleotide and second polynucleotide.

25. The method of claim 24, wherein the first and second enzymes are a pair selected from the group consisting of Not I and Bsp 120I, AscI and MluI, and SbfI and Nsi I, or isoschizomers thereof.

26. The method of claim 24, wherein steps a) through d) are repeated until the construct is complete.

27. The method of claim 24, wherein the nucleic acid construct is at least 20 kb in size.

28. The method of claim 24, wherein the nucleic acid construct is at least 30 kb in size.

29. The method of claim 24, wherein the first polynucleotide and/or the second polynucleotide comprise a coding sequence.

30. The method of claim 24, wherein the first polynucleotide and/or the second polynucleotide comprise a regulatory element.
